(19) United States
(12) Patent Application Publication
(10) Pub. No.: US 2018/0168379 A1
(43) Pub. Date: Jun. 21, 2018

(54) A HANDHELD APPARATUS WITH ORIENTATION GUIDING GRIP

(71) Applicant: Julia DELAFORCE, Macleay Island, Queensland (AU)

(72) Inventor: Julia DELAFORCE, Macleay Island, Queensland (AU)

(21) Appl. No.: 15/736,326

(22) PCT Filed: Jun. 16, 2016
(86) PCT No.: PCT/AU2016/050502

§ 371 Date: Dec. 14, 2017

(30) Foreign Application Priority Data
Jun. 16, 2015 (AU) 2015902287

(51) Int. Cl. A47G 21/04; B25G 1/10; B26B 27/00; A61F 4/00

(52) U.S. Cl. CPC A47G 21/04; A61F 4/00; B26B 27/007; B25G 1/102

(57) ABSTRACT

A handheld apparatus such as a spoon, fork or knife that is easy to hold and is less likely to be dropped by a small, weak or handicapped hand is enabled by various embodiments. Some embodiments of the apparatus include: a utensil element having a distal end and a handle end; a handle element having a distal end and a utensil end, the utensil end connected to the handle end of the utensil element; and a guard element encasing the handle element.

### BACKGROUND TO THE INVENTION

There are many situations in which using a utensil to handle or manipulate objects is necessary. For example, during eating it is common to use spoons, forks and knives to cut, scoop or pick up food. However, in many instances, this seemingly simple act can be incredibly difficult for people such as young children, the elderly, stroke victims or those afflicted by neurodegenerative diseases such as Alzheimer's disease.

Various shapes and designs have been proposed in the prior art for handheld apparatuses to make such apparatuses easier to hold. However, even such specially designed utensils are often dropped and are not easily picked up in a proper orientation by infants still lacking motor skills. Also, young children often struggle to use utensils, such as spoons and forks, effectively as they are typically unable to grasp them consistently correctly.

Using such utensils and other basic activities are often referred to as activities of daily living (ADLs). While adults typically assist young children to perform ADLs, it is expected that children develop independence with ADLs as they mature. Most children achieve the milestone ADL of being able to feed themselves simple meals using a fork or spoon by the age of three. Interestingly, feeding oneself involves utilising both fine and gross motor skills.

Parents and carers often attempt to have children learn to self-feed using large forks and spoons which are not designed for use by small children. Indeed, many standard forks and knives are metal and have potentially dangerous tines and serrated edges with which a child can pierce or cut themselves while attempting to use the utensil. Furthermore, utensils are generally made from materials that can scratch or irritate a child's mouth. Also, smaller utensils may be put into a child's mouth and have the potential to become lodged in the child's throat.

Additionally, a child may not know which end of a utensil to hold. For example, a small child, or baby, may grab a spoon at the point where the bowl of the spoon meets the handle. Grabbing the spoon in this manner prevents the child from being able to put the majority of the spoon into its mouth, resulting in mess and frustration.

Furthermore, those who suffer from neurodegenerative diseases such as Alzheimer's or Motor Neurone disease commonly suffer from the loss of their motor skills and potentially their memory of how to correctly utilise eating utensils. Additionally, those who suffer from arthritis may find it difficult to grasp an eating utensil for an extended period of time.

Accordingly, there is a need for an improved handheld apparatus with orientation guiding grip for assisting a user to independently accomplish various ADLs including but not limited to feeding oneself.

### SUMMARY OF THE INVENTION

In one form, although not necessarily the only or the broadest form, the invention resides in a handheld apparatus comprising:

a utensil element having a distal end and a handle end; and

a handle element having a distal end and a utensil end, the utensil end connected to the handle end of the utensil element;

wherein the handle element comprises a grip material that is plastically deformable and settable, such that in use the grip material plastically deforms when gripped by a human hand and then hardens to retain an impression of the human hand.

Preferably, the utensil element defines one of a spoon, fork or knife.

Preferably, the handle element comprises an elongate core surrounded by the grip material.

Preferably, the grip material comprises one of a thermoset polymer material, clay, or other material.

Preferably the elongate core of the handle element is integrally formed with the utensil element and is detachable.

In another form, although not necessarily the only or the broadest form, the invention resides in a handheld apparatus comprising:

a utensil element having a distal end and a handle end;

a handle element having a distal end and a utensil end, the utensil end connected to the handle end of the utensil element; and

a guard element encasing the handle element.

Preferably, the guard element defines a first aperture for receiving a human hand.

Preferably, the guard element defines a second aperture for receiving the handle element.

Preferably, the guard element comprises a socket that is positioned opposite the second aperture and the socket receives the distal end of the handle element.

Preferably, the socket restricts rotation of the handle element in the second aperture.

Preferably, the second aperture defines a socket that restricts rotation of the handle element in the second aperture.

Preferably, the second aperture is positioned approximately ninety degrees from the first aperture along a surface of the guard element.

Preferably, the socket defines a discrete number of rotational positions of a longitudinal axis of the utensil element relative to the guard element.

Preferably, a shape of the first aperture for receiving a human hand is generally elliptical.

Preferably, the guard element defines a spheroid.

Preferably, the handle element is removable from the guard element.

Preferably, the utensil element defines one of a spoon, fork and a knife.

In another form, although not necessarily the only or the broadest form, the invention resides in a kit for a handheld apparatus comprising:

the handheld apparatus as defined above; and

a plurality of alternative utensil elements.

Preferably, the plurality of alternative utensil elements define more than one of a spoon, a fork and a knife.

Preferably, the utensil element and the handle element are integrally formed.

In another form, although not necessarily the only or the broadest form, the invention resides in a handheld apparatus comprising:

a utensil element having a distal end and a handle end;

a handle element having a distal end and a utensil end, the utensil end connected to the handle end of the utensil element; and

a strap removably attachable to the distal end of the handle element and connected to the handheld apparatus between the distal end of the handle element and the distal end of the utensil element.

Preferably, the utensil element defines one of a spoon, fork and a or other utensils.

Preferably, the strap includes holes for receiving the distal end of the handle element.

Preferably, the strap can be manufactured in varying sizes and widths, and can include holes of varying sizes and widths.

Preferably, the strap is connected to the utensil end of the handle element.

Preferably, the strap comprises an elastomeric material, silicon, sugru, paper, or rayon.

### BRIEF DESCRIPTION OF THE DRAWINGS

By way of example only, preferred embodiments of the invention will be described more fully hereinafter with reference to the accompanying figures, wherein:

FIG. 1 shows a perspective view of a handheld apparatus according to an embodiment of the present invention.

FIG. 2 shows a side view of the handheld apparatus of FIG. 1.

FIG. 3 shows an underside view of the handheld apparatus of FIG. 1.

FIG. 4 shows a top view of the handheld apparatus of FIG. 1.

FIG. 5 shows a perspective view of a utensil according to an alternative embodiment of the present invention.

FIG. 6 shows a perspective view of a guard element according to an embodiment of the present invention.

FIG. 7 shows a perspective view of a handheld apparatus according to an embodiment of the present invention, including the utensil of FIG. 5 inserted into the guard element of FIG. 6.

FIG. 8 shows an underside view of the handheld apparatus of FIG. 7.

FIG. 9 shows a side view of the handheld apparatus of FIG. 7.

FIG. 10 shows a top view of the handheld apparatus of FIG. 7.

FIG. 11 shows a rear end view of the handheld apparatus of FIG. 7.

FIG. 12 shows a front end view of the handheld apparatus of FIG. 7.

FIG. 13 shows a perspective view of a handheld apparatus according to another alternative embodiment of the present invention.

FIG. 14 shows a side view of the handheld apparatus of FIG. 13.

FIG. 15 shows an overhead view of the handheld apparatus of FIG. 13.

FIG. 16 shows an underside view of the handheld apparatus of FIG. 13.

Those skilled in the art will appreciate that minor deviations from the symmetrical layout of components as illustrated in the drawings will not detract from the proper functioning of the disclosed embodiments of the present invention.

### DETAILED DESCRIPTION OF THE PREFERRED EMBODIMENT

The present invention relates to a handheld apparatus having an orientation guiding grip. Elements of the invention are illustrated in concise outline form in the drawings, showing only those specific details that are necessary to understand the embodiments of the present invention, but so as not to clutter the disclosure with excessive detail that will be obvious to those of ordinary skill in the art in light of the present description.

In this patent specification, adjectives such as first and second, left and right, front and back, top and bottom, etc., are used solely to define one element or method step from another element or method step without necessarily requiring a specific relative position or sequence that is described by the adjectives. Words such as “comprises” or “includes” are not used to define an exclusive set of elements or method steps. Rather, such words merely define a minimum set of elements or method steps included in a particular embodiment of the present invention.

According to a first aspect, the present invention is defined as a handheld apparatus. The handheld apparatus comprises a utensil element having a distal end and a handle end. The handheld apparatus further comprises a handle element having a distal end and a utensil end. The utensil end of the handle element is connected to the handle end of the utensil element. Further, the handle element comprises a grip material that is plastically deformable and settable. In use, the grip material plastically deforms when gripped by a human hand and then hardens to retain an impression of the human hand.

According to a second aspect, the present invention is defined as a handheld apparatus comprising a utensil element having a distal end and a handle end. The handheld apparatus further comprises a handle element having a distal end and a utensil end. The utensil end of the handle element is connected to the handle end of the utensil element. The handheld apparatus additionally comprises a guard element encasing the handle element.

According to a third aspect, the present invention is defined as a handheld apparatus comprising a utensil element having a distal end and a handle end. The handheld apparatus further comprises a handle element having a distal end and a utensil end, the utensil end is connected to the handle end of the utensil element. The handheld apparatus further comprises a strap removably attachable to the distal end of the handle element and connected to the handheld apparatus between the distal end of the handle element and the distal end of the utensil element.

Advantages of embodiments of the present invention include a handheld apparatus which is easy to hold and cannot be easily dropped.

Also, embodiments of the present invention can effectively guide a user's hand to an appropriate gripping orientation relative to the apparatus.

Further advantages of embodiments of the present invention include the ability to disassemble the handheld apparatus and interchange different types of utensils, as required.

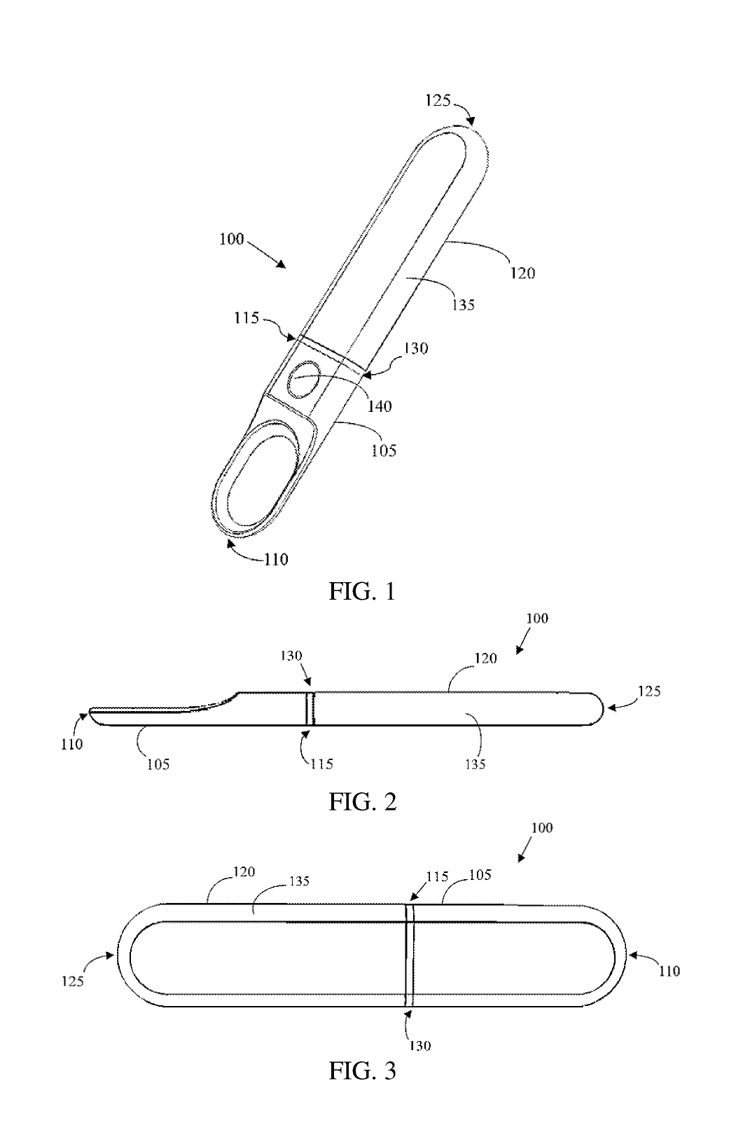

FIG. 1 shows a perspective view of a handheld apparatus 100 comprising a utensil element 105 having a distal end 110 and a handle end 115. Some embodiments of the utensil element 105 comprise one of a spoon, fork or knife or other elements.

The handheld apparatus 100 further comprises a handle element 120 having a distal end 125 and a utensil end 130. The utensil end 130 of the handle element 120 is connected to the handle end 115 of the utensil element 105. Further, the handle element 120 comprises a grip material 135 that is plastically deformable and settable. The grip material 135 can be one of a thermoset polymer material, a clay, sugru or any suitable material which can be moulded. Advantageously, the thermoset polymer material can be a food grade ethylene-vinyl acetate. In use, the grip material 135 plastically deforms when gripped by a human hand (not shown) and then hardens to retain an impression of the human hand. For example, similar to the conditioning process for mouth guards, where a mouth guard is first softened by placing it in boiling water and then is custom fit to a user's mouth, the handle element 120 of the present invention can be custom fit to a user's hand once and then used repeatedly.

Some embodiments of the handle element 120 include an elongate core (not shown) which is surrounded by the grip material 135. In use, the utensil element 105 is able to be disengaged from the handle element 120. In some alternative embodiments of the handheld apparatus 100, the elongate core is integrally formed with the utensil element 105. The utensil element 105 may further comprise a depression 140 for assisting with separating the handle element 105 from the handle element 120.

FIG. 2 illustrates a side view of the handheld apparatus 100.

FIG. 3 illustrates an underside view of the handheld apparatus 100.

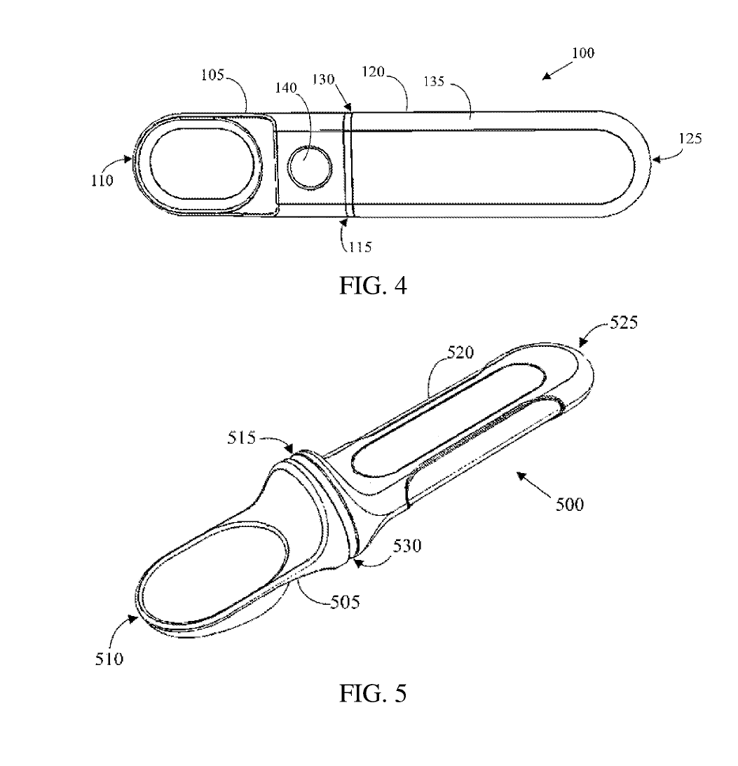

FIG. 4 illustrates a top view of the handheld apparatus 100.

The utensil element 105 may comprise various materials such as polypropylene or plastic materials. However, as will be appreciated by those skilled in the art, the utensil element 105 can be varied to suit particular requirements as demanded by a particular application. Advantageously, a polypropylene utensil element can help prevent a user from cutting or irritating the delicate lining of the mouth.

FIG. 5 illustrates a perspective view of a utensil 500 comprising a utensil element 505 having a distal end 510 and a handle end 515. For example, the utensil element 505 may comprise at least one of a fork, spoon and a knife. Similar to the apparatus 100, the utensil element 505 may comprise polypropylene or plastic materials. Advantageously, a polypropylene utensil element can be manufactured to be dishwasher safe, hypoallergenic and free from Bisphenol A (BPA), Bisphenol S (BPS), Phthalates and PVC.

The handheld apparatus 500 further comprises a handle element 520 having a distal end 525 and a utensil end 530. The utensil end 530 of the handle element 520 is connected to the handle end 515 of the utensil element 505.

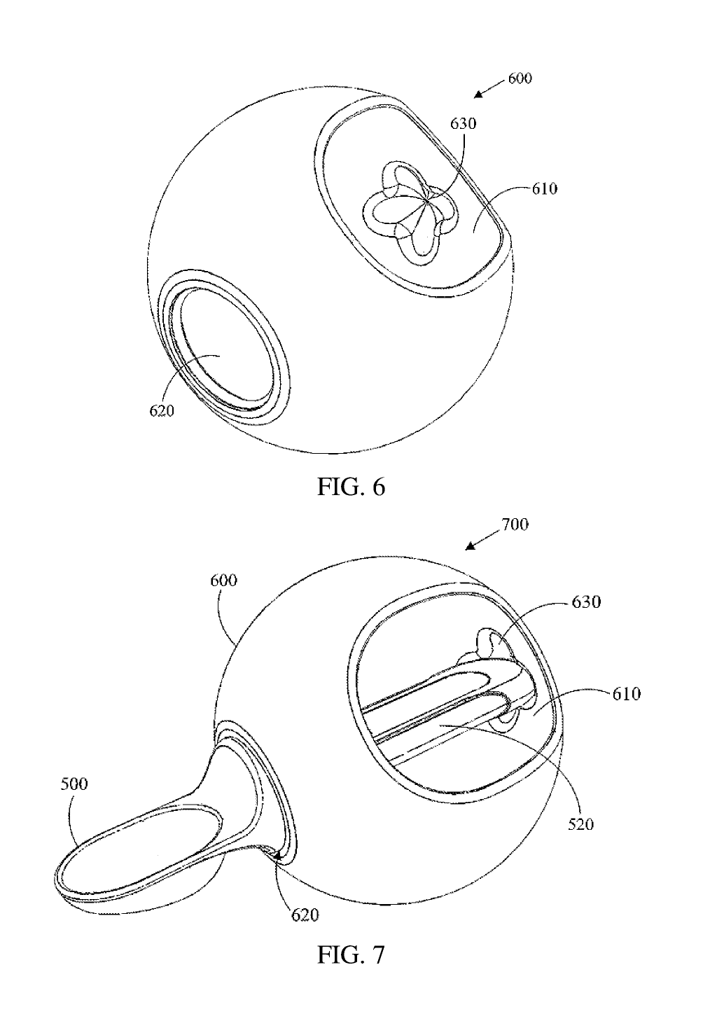

FIG. 6 illustrates a perspective view of a guard element 600 for encasing the handle element 520 of the handheld apparatus 500 shown in FIG. 5. In some embodiments of the invention, the guard element 600 defines a spheroid. However, it will be appreciated by a person skilled in the art that the guard element 600 can define any polyhedral shape. The guard element 600 may also comprise various materials such as thermoplastic elastomer, silicon or rubber materials (such as flexible food grade silicon that is latex free). Advantageously, such materials provide a guard element that is difficult to break if dropped. Further advantageously, soft rubber and silicon materials are less likely to irritate the skin of a human hand.

The guard element 600 defines a first aperture 610 for receiving a human hand (not shown). The guard element 600 also defines a second aperture 620 for receiving the handle element 520 of the utensil 500. The second aperture 620 can be positioned, for example, approximately ninety degrees from the first aperture 610 along a surface of the guard element 600. As shown, the shape of the first aperture 610 for receiving a human hand may be generally elliptical. However, it will be appreciated by a person skilled in the art that the shape of the first aperture 610 for receiving a human hand can be varied. For example, the shape of the first aperture 610 also can include any polygonal shape suitable for receiving a human hand. Some embodiments of the guard element 600 may comprise further apertures for receiving a second human hand such as an adult's hand for assisting a child in using the guard element 600 in combination with the utensil 500. Furthermore, the guard element 600 may comprise a socket 630 that is positioned opposite the second aperture 620, and where the socket 630 receives the distal end 525 of the handle element 520 of the utensil 500.

FIG. 7 illustrates a perspective view of a handheld apparatus 700 comprising the utensil 500 and the guard element 600 assembled together. In use, the first aperture 610 receives a human hand (not shown) which grips the handle element 520. The handle element 520 may be removable from the guard element 600 for cleaning or for substituting alternative utensils.

In some embodiments, the socket 630 restricts rotation of the handle element 520. As shown, some embodiments of the guard element 600 of the handheld apparatus 500 include the socket 630 which defines a discrete number of rotational positions of a longitudinal axis of the utensil element 505 relative to the guard element 600. The longitudinal axis extends along a line (not shown) connecting the distal end 510 of the utensil element 505 and the distal end 525 of the handle element 520. As shown, the “+” shape of the socket 630 enables four different rotational positions of the utensil 500, relative to the guard element 600, each spaced ninety degrees apart. As will be understood by those skilled in the art, according to alternative embodiments a greater or lesser number of optional rotational positions can be provided.

The handheld apparatus 700 may be manufactured, for example, by extrusion moulding, rotation moulding, injection moulding or thermoforming.

FIG. 8 illustrates an underside view of the handheld apparatus 500. As shown, the guard element 600 of the handheld apparatus 500 may further include a portion 810 in the surface of the guard element 600 so that the surface of the guard element 600 sits flush with a second surface (not shown) such as the surface of a table and can help prevent the guard element 600 from rolling.

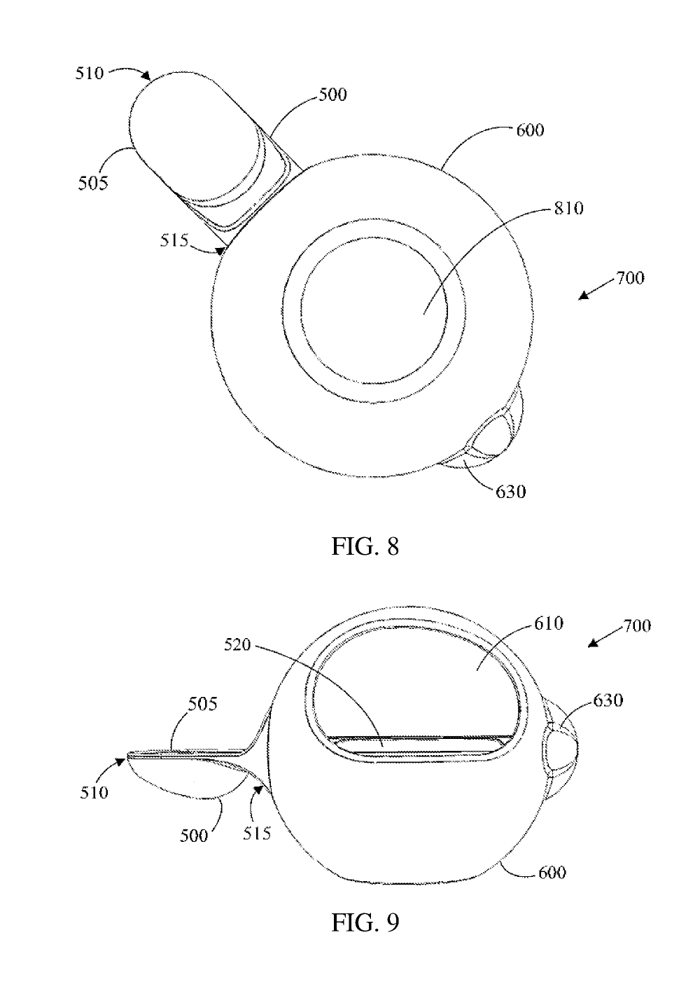

FIG. 9 shows a side view of the handheld apparatus 700. This view further illustrates the substantially flat nature of the portion 810 on the surface of the guard element 600 for sitting flush with a second surface (not shown).

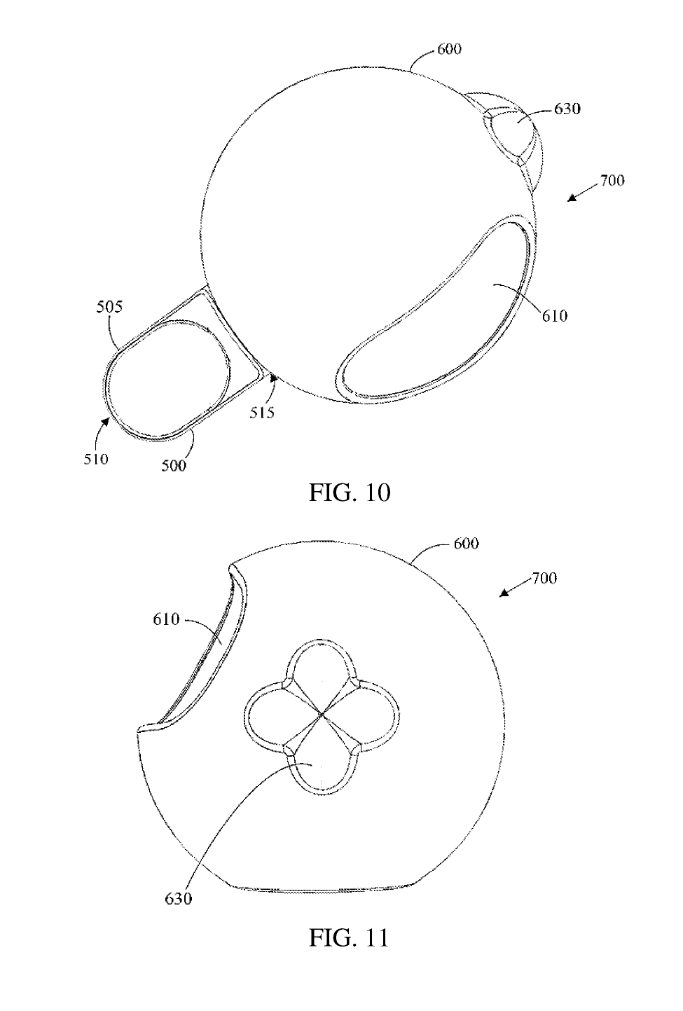

FIG. 10 shows an overhead view of the handheld apparatus 700.

FIG. 11 shows a rear end view of the handheld apparatus 700. This view further illustrates the socket 630. As shown, the socket 630 defines four rotational positions of a longitudinal axis of the utensil element 505.

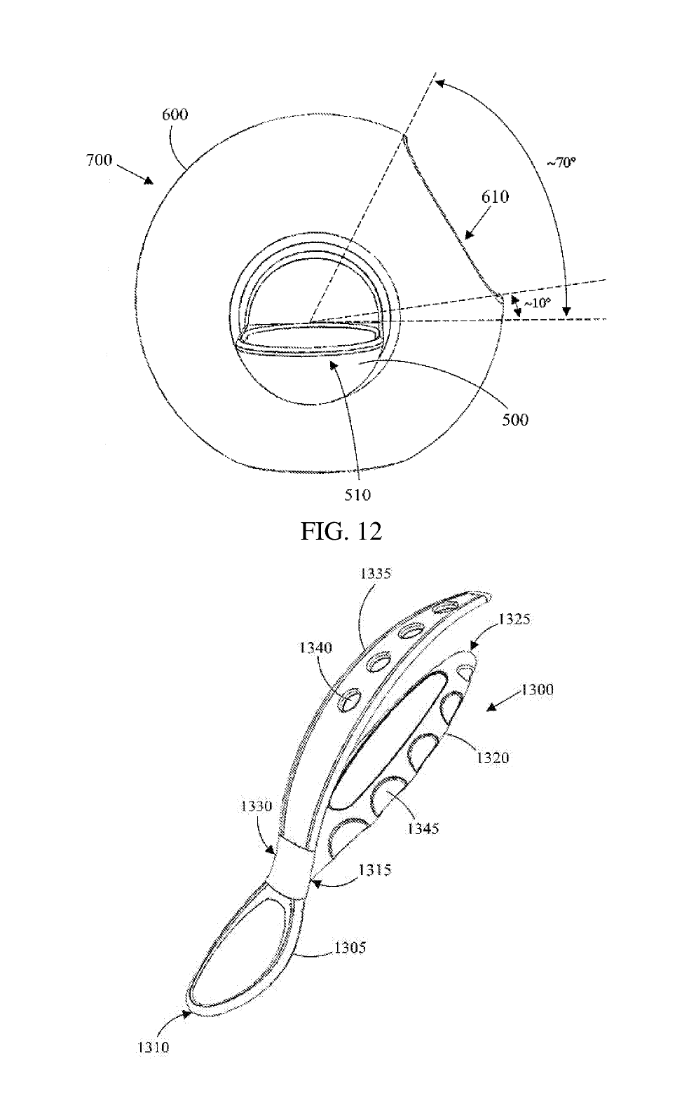

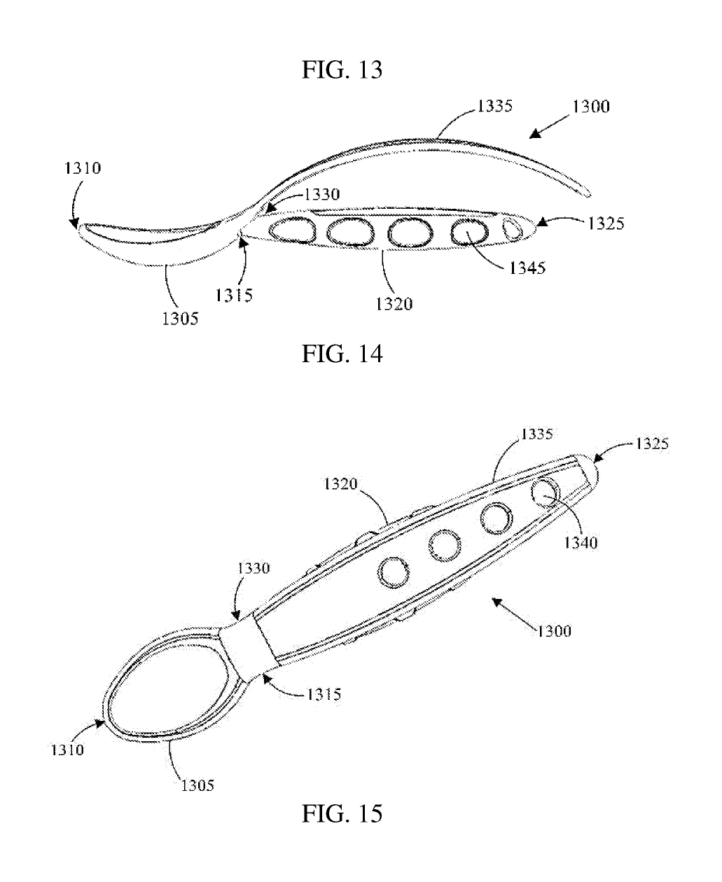

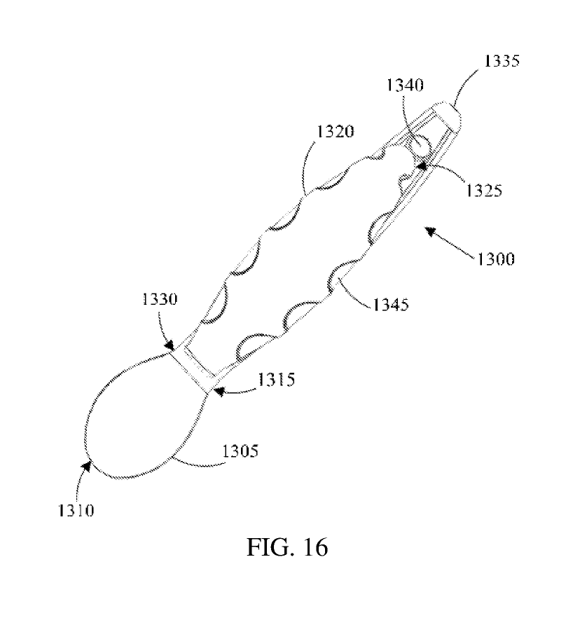

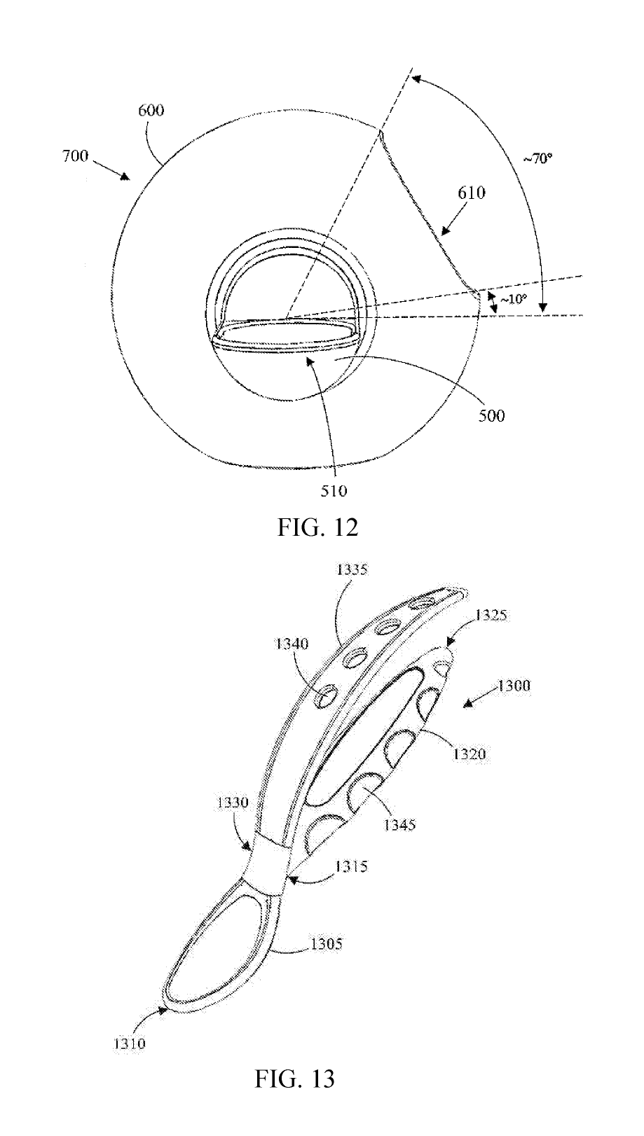

FIG. 12 shows a front end view of the handheld apparatus 700. As shown, the first aperture 610 extends between approximately 10 degrees and 70 degrees from a horizontal plane (as for example defined by a plane positioned across the portion 810, or by a plane positioned across the upper edges of the spoon of the utensil 500). The use of such angles generally provides for an appropriate and comfortable position of a human arm extending outward from the first aperture 610, and can assist in teaching, for example, a baby how to properly hold a spoon or fork relative to a plate of food. Other utensils, such as a knife, can require the use of larger angles, which can be achieved by changing the rotational position of the distal end 525 of the handle element 520 by 90 degrees in the socket 630.

FIG. 13 shows a perspective view of a handheld apparatus 1300 comprising a utensil element 1305 having a distal end 1310 and a handle end 1315. Some embodiments of the utensil element 1305 comprise one of a spoon, fork or knife. The utensil element 1305 can comprise, for example, polypropylene or plastic materials.

The handheld apparatus 1300 further comprises a handle element 1320 having a distal end 1325 and a utensil end 1330. The utensil end 1330 is connected to the handle end 1315 of the utensil element 1305. The handheld apparatus 1300 further comprises a strap 1335 removably attachable to the distal end 1325 of the handle element 1320 and connected to the handheld apparatus 1300 between the distal end 1325 of the handle element 1320 and the distal end 1310 of the utensil element 1305. The strap 1335 also can comprise, for example, thermoplastic, elastomer or silicon materials. Advantageously, such materials can provide a strap that is easy to manipulate and stretch. Further advantageously, soft rubber and silicon materials are less likely to irritate the skin of a human hand.

In some embodiments, the strap 1335 includes holes 1340 for receiving the distal end 1325 of the handle element 1320. Advantageously, the strap 1335 may secure the handheld apparatus 1300 to a human hand (not shown). In use, a human hand grips the handle element 1320 and the strap 1335 and secures the handheld apparatus 1300 to the human hand when the distal end 1325 of the handle element 1320 is inserted through one of the holes 1340 of the strap 1335. In some alternative embodiments, the strap 1335 is connected to the utensil end 1330 of the handle element 1320, or one end of the strap 1335 fits like a glove over the utensil element 1305, thus enabling the strap 1335 to be used with a conventional utensil element such as a spoon.

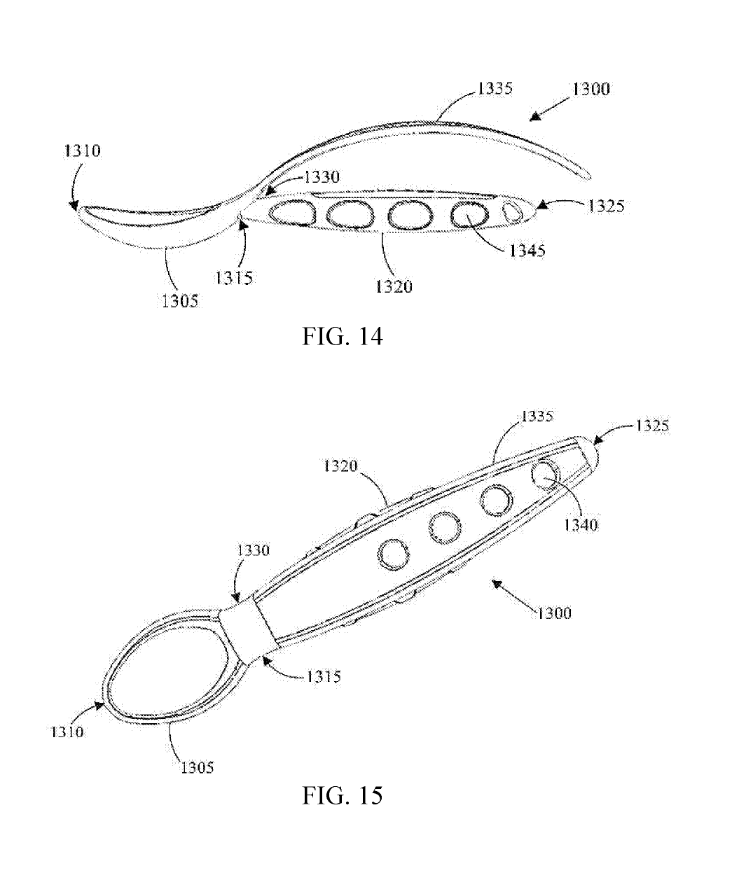

FIG. 14 illustrates a side view of the handheld apparatus 1300. As shown, the handheld apparatus 1300 may further include impressions 1345 for guiding the placement of the fingers of a human hand (not shown). Further, the strap 1335 may be curved to assist in securing the strap 1335 to the distal end 1325 of the handle element 1320.

FIG. 15 shows an overhead view of the handheld apparatus 1300.

FIG. 16 illustrates a view of the underside of the handheld apparatus 1300.

In summary, advantages of some embodiments of the present invention include a handheld apparatus that is easy to hold and less likely to be dropped by a small, weak or handicapped hand. Also, embodiments of the present invention can effectively guide a user's hand to an appropriate gripping orientation relative to the apparatus. Further, some embodiments of the present invention are able to be moulded to fit the contours of a human hand. Other advantages include a handheld apparatus that can be fastened to a human hand to prevent dropping of the apparatus. Further, the soft properties of the materials of the elements of the apparatus lessen the chance that the lining of a user's mouth will be irritated or damaged while using the handheld apparatus. It will be understood by the skilled person that all of the above advantages are not necessarily applicable to all embodiments of the present invention.

The above description of various embodiments of the present invention is provided for purposes of description to one of ordinary skill in the related art. It is not intended to be exhaustive or to limit the invitation to a single disclosed embodiment. Numerous alternatives and variations to the present invention will be apparent to those skilled in the art of the above teaching. Accordingly, this patent specification is intended to embrace all alternatives, modifications and variations of the present invention that have been discussed herein, and other embodiments that fall within the spirit and scope of the above described invention.

1. A handheld apparatus, comprising:
a utensil element having a distal end and a handle end;
a handle element having a distal end and a utensil end, the utensil end connected to the handle end of the utensil element; and
a guard element encasing the handle element.

2. The handheld apparatus of claim 1, wherein the guard element defines a first aperture for receiving a human hand.

3. The handheld apparatus of claim 2, wherein the guard element defines a second aperture for receiving the handle element.

4. The handheld apparatus of claim 3, wherein the guard element comprises a socket that is positioned opposite the second aperture and the socket receives the distal end of the handle element.

5. The handheld apparatus of claim 4, wherein the socket restricts rotation of the handle element in the second aperture.

6. The handheld apparatus of claim 3, wherein the second aperture defines a socket that restricts rotation of the handle element in the second aperture.

7. The handheld apparatus of claim 2, wherein the second aperture is positioned approximately ninety degrees from the first aperture along a surface of the guard element.

8. The handheld apparatus of claim 4, wherein the socket defines a discrete number of rotational positions of a longitudinal axis of the utensil element relative to the guard element.

9. The handheld apparatus of claim 1, wherein a shape of the first aperture for receiving a human hand is generally elliptical.

10. The handheld apparatus of claim 1, wherein the guard element defines a spheroid.

11. The handheld apparatus of claim 1, wherein the handle element is removable from the guard element.

12. The handheld apparatus of claim 1, wherein the utensil element defines one of a spoon, fork and a knife.

13. The handheld apparatus of claim 1, wherein the utensil element and the handle element are integrally formed.

14. A kit for a handheld apparatus, the kit comprising:
the handheld apparatus of claim 1; and
a plurality of alternative utensil elements.

15. The kit of claim 14, wherein the plurality of alternative utensil elements define more than one of a spoon, a fork and a knife.

16. A handheld apparatus, comprising:
a utensil element having a distal end and a handle end;
a handle element having a distal end and a utensil end, the utensil end connected to the handle end of the utensil element; and
a strap removably attachable to the distal end of the handle element and connected to the handheld apparatus between the distal end of the handle element and the distal end of the utensil element.

17. The handheld apparatus of claim 16, wherein the utensil element defines one of a spoon, fork and a knife.

18. The handheld apparatus of claim 16, wherein the strap includes holes for receiving the distal end of the handle element.

19. The handheld apparatus of claim 16, wherein the strap is connected to the utensil end of the handle element.

20. The handheld apparatus of claim 16, wherein the strap comprises an elastomeric material.

21. (canceled)

22. (canceled)

23. (canceled)

24. (canceled)

25. (canceled)
